(19) United States
(12) Patent Application Publication
(10) Pub. No.: US 2011/0197496 A1
(43) Pub. Date: Aug. 18, 2011

(54) PRODUCING FUEL AND SPECIALTY CHEMICALS FROM GLYCERIDE CONTAINING BIOMASS

(75) Inventors: Paul O'Connor, Km Hoevelaken (NL); Steve Yanik, Colorado Springs, CO (US)

(73) Assignee: KIOR, INC., Pasadena, TX (US)

(21) Appl. No.: 12/997,284

(22) PCT Filed: Jun. 30, 2009
(86) PCT No.: PCT/US2009/049247

§ 371 Date: Apr. 21, 2011

Related U.S. Application Data
(60) Provisional application No. 61/076,966, filed on Jun. 30, 2008.

(51) Int. Cl. C10L 1/04; C07C 1/20

(52) U.S. Cl. 44/307; 585/310

(57) ABSTRACT

A method for catalytically cracking a triglyceride-containing biomass can include the steps of (i) catalytically cracking triglycerides in a biomass-catalyst mixture, at a temperature below about 300° C., to produce a first oil and a cellulosic portion and (ii) catalytically cracking cellulose in the cellulosic portion, at a temperature between about 300 and about 600° C., to produce a second oil. Another method for catalytically cracking a triglyceride-containing biomass includes catalytically cracking triglycerides in a biomass-catalyst mixture, at a temperature between about 300 and about 600° C., where the catalyst comprises an acidic or basic catalyst, to produce an oil comprising aromatics.

### FIELD OF THE INVENTION

The invention relates generally to the production of fuel and specialty chemicals from biomass. The invention relates, more particularly, to the production of diesel fuel, jet fuel, and specialty chemicals from triglyceride containing biomass.

### BACKGROUND OF THE INVENTION

Biomass, in particular biomass of plant origin, is recognized as an abundant potential source of fuels and specialty chemicals. See, for example, “Energy production from biomass,” by P. McKendry—Bioresource Technology 83 (2002) p 37-46 and “Coordinated development of leading biomass pretreatment technologies” by Wyman et al., Bioresource Technology 96 (2005) 1959-1966. Refined biomass feedstock, such as vegetable oils, starches, and sugars, can be substantially converted to liquid fuels including biodiesel (e.g., methyl or ethyl esters of fatty acids) and ethanol. However, using refined biomass feedstock for fuels and specialty chemicals can divert food sources from animal and human consumption, raising financial and ethical issues.

Alternatively, triglyceride-containing biomass, such as aquatic biomass, can be used to produce liquid fuels and specialty chemicals. In many cases, triglyceride containing biomass does not presently have an economic use. Examples of such biomass include micro and macro algae, other aquatic plants, oil seed, and the like. Such biomass generally includes lipids (e.g., glycerides and aliphatic hydrocarbons), as well as carbohydrates (e.g., lignin, amorphous hemi-cellulose, and crystalline cellulose) and polypeptides (e.g., proteins).

However, converting triglyceride containing biomass to fuel and specialty chemicals presents numerous challenges. For example, conversion of such biomass traditionally proceeds through separation (e.g., of triglyceride from fibrous components), steam reforming, oligomerization, and isomerization of the triglycerides, and finally production of the end product. Separation can reduce product yield because triglycerides can be lost to the fibrous component, which must be separately converted or removed as waste. Separation can also require a large input of energy (e.g., for disintegrating and separating the components, and for steam reforming, which requires temperatures greater than about 800° C.). Furthermore, the efficiency of converting the biomass can also be reduced by the extreme conditions (e.g., elevated temperature and/or pressure), which necessitates oligomerization and isomerization steps to build up the molecules after they have been broken down by the extreme conditions. Such biomass can be difficult to convert because different organic compounds (e.g., lipids, carbohydrates, and amino acids) may require different conditions for high-yield and efficient conversion. In various traditional conversion processes, only up to about 50% of the triglyceride containing biomass is converted into fuel and/or specialty chemicals. Therefore, higher yielding and more efficient methods and apparatuses for converting triglyceride containing biomass to fuel and specialty chemicals are needed to make triglyceride containing biomass a viable feedstock.

### BRIEF SUMMARY OF THE INVENTION

The invention includes methods, apparatuses, kits, and compositions for converting triglyceride containing biomass, including aquatic biomass, into fuels and/or specialty chemicals under conditions that mitigate equipment cost, energy cost, unwanted degradation of conversion products, and undesirable reactions of conversion products. Examples of fuels include light gases (ethane, propane, butane), naphtha, and distillates (jet fuel, diesel, heating oil). Examples of chemicals include light olefins (ethylene, propylene, butylenes), acids (like formic and acetic), aldehydes, alcohols (ethanol, propanol, butanol, phenols), ketones, furans, and the like. Thus, the invention reduces the cost and increases the availability of fuel and/or specialty chemicals derived from triglyceride containing biomass. The invention also increases the value and utility of triglyceride containing biomass feedstock for fuel and/or specialty chemical production.

The invention includes catalytic conversion of biomass, which can improve conversion of the biomass components, including triglycerides, into fuels and/or specialty chemicals. The invention includes methods using conventional petroleum refining process units (e.g., known petrochemical refining units), adapting existing refinery units for processing biomass (e.g., adapting operating parameters and feedstock), converting existing refinery process units for processing biomass, and constructing new, customized biomass reactors (e.g., employing commercially available conventional petroleum reactor components and/or customized components). Preparation can have a synergistic effect, reducing the temperature necessary for catalytic conversion of the biomass and/or increasing the conversion efficiency of the biomass, as well as facilitating processing in conventional petroleum refining process units.

In one aspect, the invention features a method for producing high-quality JP-8 or diesel fuel from a triglyceride-rich biomass such as algae or oil seed. The method includes catalytically cracking triglycerides in a triglyceride-rich biomass-catalyst mixture, at a temperature below about 300° C., to produce a first bio-oil and a cellulosic residue. The method also includes catalytically cracking cellulose in the cellulosic residue, at a temperature between about 300 and about 600° C., to produce a second bio-oil. Furthermore, the method includes mildly hydro-cracking the second bio-oil and, optionally, mildly hydrocracking at least a portion of the first bio-oil, to produce a hydro-cracked portion. The method also includes isomerizing or alkylating the hydro-cracked portion and, optionally isomerizing or alkylating at least a portion of the first bio-oil, to produce high-quality JP-8 or diesel fuel.

In another aspect, the invention features a method for producing high-quality JP-8 or diesel fuel from a triglyceride-rich biomass such as algae or oil seed. The method includes catalytically cracking triglycerides in a triglyceride-rich biomass-catalyst mixture, at a temperature below about 300° C., to produce a first bio-oil and a cellulosic residue. The method also includes catalytically cracking cellulose in the cellulosic residue, at a temperature between about 300 and about 600° C., together with the first bio-oil and an acidic or basic catalyst, to produce a second bio-oil including aromatics. Furthermore, the method includes mildly hydro-cracking the second bio-oil, to produce high-quality JP-8 or diesel fuel.

In still another aspect, the invention features a method for producing high-quality JP-8 or diesel fuel from a triglyceride-rich biomass such as algae or oil seed. The method includes catalytically cracking triglycerides in a triglyceride-rich biomass-catalyst mixture, at a temperature between about 300 and about 600° C., to produce a bio-oil including aromatics. The catalyst includes an acidic catalyst. The method also includes mildly hydro-cracking the bio-oil, to produce high-quality JP-8 or diesel fuel.

In yet another aspect, the invention features a method for catalytically cracking a triglyceride-containing biomass. The method includes catalytically cracking triglycerides in a biomass-catalyst mixture, at a temperature below about 300° C., to produce a first oil and a cellulosic portion. The method also includes catalytically cracking cellulose in the cellulosic portion, at a temperature between about 300 and about 600° C., to produce a second oil.

In one aspect, the invention features a method for catalytically cracking a triglyceride-containing biomass. The method includes catalytically cracking triglycerides in a biomass-catalyst mixture, at a temperature between about 300 and about 600° C., to produce an oil including aromatics. The catalyst includes an acidic or basic catalyst.

In another aspect, the invention features a composition for producing fuel from a triglyceride-containing biomass. The composition includes a biomass-catalyst mixture. The biomass includes triglycerides and cellulosic particles. The catalyst includes a solid particulate catalyst and the biomass-catalyst mixture includes at least a portion of the catalyst mechano-chemically interacting with at least a portion of the cellulosic particles. Alternatively, the catalyst is capable of being at least partly dissolved or suspended in a liquid and the biomass-catalyst mixture includes at least a portion of the catalyst impregnating at least a portion of the cellulosic particles.

In still another aspect, the invention features a method for converting a conventional petroleum petrochemical refinery process for catalytically cracking a triglyceride-containing biomass. The method includes providing or adapting a first conventional petroleum reactor for catalytically cracking triglycerides in a biomass-catalyst mixture, at a temperature below about 300° C., to produce a first oil and a cellulosic portion. The method also includes providing or adapting the second conventional petroleum reactor for catalytically cracking cellulose in the cellulosic portion, at a temperature between about 300 and about 600° C., to produce a second oil.

In yet another aspect, the invention features a kit for converting a conventional petroleum petrochemical refinery process for catalytically cracking a triglyceride-containing biomass. The kit includes instructions for adapting a first reactor for catalytically cracking triglycerides in a biomass-catalyst mixture, at a temperature below about 300° C., to produce a first oil and a cellulosic portion. The kit also includes instructions for adapting a second reactor for catalytically cracking cellulose in the cellulosic portion, at a temperature between about 300 and about 600° C., to produce a second oil.

In one aspect, the invention features a kit for converting a conventional petroleum petrochemical refinery process for catalytically cracking a triglyceride-containing biomass comprising. The kit includes a first reactor labeled for catalytically cracking triglycerides in a biomass-catalyst mixture, at a temperature below about 300° C., to produce a first oil and a cellulosic portion. The kit also includes a second reactor labeled for catalytically cracking cellulose in the cellulosic portion, at a temperature between about 300 and about 600° C., to produce a second oil.

In other examples, any of the aspects above, or any method, apparatus, or composition of matter described herein, can includes one or more of the following features.

In various embodiments, methods include separating the first oil from the cellulosic portion before catalytically cracking cellulose in the cellulosic portion, and hydro-cracking the first oil and the second oil to produce a hydro-cracked product. Methods can include separating the first oil from the cellulosic portion before catalytically cracking cellulose in the cellulosic portion, and hydro-cracking the second oil, to produce a hydro-cracked product. Methods can include catalytically cracking cellulose in the cellulosic residue, together with the first bio-oil and an acidic or basic catalyst, to produce a second bio-oil including aromatics.

In some embodiments, methods include isomerizing or alkylating the hydro-cracked product, to produce a fuel or specialty chemical. Methods can include isomerzing the first oil and the hydro-cracked product, to produce a fuel or specialty chemical. Methods can include hydro-cracking the second oil, to produce a fuel or specialty chemical. Methods can include hydro-cracking the oil, to produce a fuel or specialty chemical. At least a portion of the hydro-cracked product can be used to produce a fuel or specialty chemical. The fuel or specialty chemical can include JP-8 or diesel fuel.

In certain embodiments, the biomass includes algae. The biomass can include oil seed.

In various embodiments, the catalyst includes a solid particulate catalyst and the biomass-catalyst mixture includes at least a portion of the catalyst mechano-chemically interacting with at least a portion of the solid biomass particles.

In some embodiments, the catalyst includes a catalyst capable of being at least partly dissolved or suspended in a liquid and the biomass-catalyst mixture includes at least a portion of the catalyst impregnating at least a portion of the solid biomass particles.

In certain embodiments, the triglycerides include cracked triglycerides.

In various embodiments, the catalyst includes an acidic catalyst. The catalyst can be a basic catalyst.

In some embodiments, kits and/or apparatuses include a system for separating the first oil from the cellulosic portion and for providing the first oil for mild hydro-cracking or isomerization. The second conventional petroleum reactor can be adapted for reacting the first oil, with an acidic or basic catalyst, to produce aromatics. Kits and/or apparatuses can include instructions for adapting a conventional petroleum hydro-cracking reactor for deoxygenating at least one of the first oil and the second oil, to produce a deoxygenated product.

In certain embodiments, kit and/or apparatuses include a system for separating at least a portion of the deoxygenated product, to produce at least one of a fuel, specialty chemical, JP-8, or diesel. Kits and/or apparatuses can include instructions for adapting a conventional petroleum isomerizing or alkylating reactor for isomerizing or alkylating at least one of the first oil and the deoxygenated product, to produce at least one of a fuel, specialty chemical, JP-8, or diesel.

In various embodiments, a method can include catalytically cracking any one or more of a protein, nucleic acid, lignin or other non-cellulosic carbohydrate, and non-triglyceride lipid.

Other aspects and advantages of the invention will become apparent from the following drawings and description, all of which illustrate principles of the invention, by way of example only.

### BRIEF DESCRIPTION OF THE DRAWINGS

The advantages of the invention described above, together with further advantages, may be better understood by referring to the following description taken in conjunction with the accompanying drawings. The drawings are not necessarily to scale, emphasis instead generally being placed upon illustrating the principles of the invention.

FIG. 1 shows a method for catalytically cracking a triglyceride-containing biomass including two catalytic cracking steps and an isomerization step.

FIG. 2 shows a method for catalytically cracking a triglyceride-containing biomass including two catalytic cracking steps.

FIG. 3 shows a method for catalytically cracking a triglyceride-containing biomass including one catalytic cracking step.

FIG. 4 shows a method for processing a triglyceride-containing biomass without an oil extraction step.

FIG. 5 shows a method for co-processing a refinery feedstock and a triglyceride-containing biomass without an oil extraction step.

### DETAILED DESCRIPTION OF THE INVENTION

The invention provides methods, apparatuses, kits, and compositions for converting triglyceride material in biomass into fuels and/or specialty chemicals under conditions that can mitigate equipment cost, energy cost, and/or degradation or undesirable reaction of conversion product.

The invention provides methods for catalytically cracking a triglyceride-containing biomass. In one example, a method can include catalytically cracking triglycerides in a biomass-catalyst mixture, at a temperature below about 300° C., to produce a first oil and a cellulosic portion. The method also includes catalytically cracking cellulose in the cellulosic portion, at a temperature between about 300 and about 600° C., to produce a second oil. In another example, a method includes catalytically cracking triglycerides in a biomass-catalyst mixture, at a temperature between about 300 and about 600° C., to produce an oil including aromatics. The catalyst can include an acidic or basic catalyst.

The invention also provides compositions for producing fuel from a triglyceride-containing biomass. The composition includes a biomass-catalyst mixture. The biomass includes triglycerides and cellulosic particles. The catalyst includes a solid particulate catalyst and the biomass-catalyst mixture includes at least a portion of the catalyst mechano-chemically interacting with at least a portion of the cellulosic particles. Alternatively, the catalyst is capable of being at least partly dissolved or suspended in a liquid and the biomass-catalyst mixture includes at least a portion of the catalyst impregnating at least a portion of the cellulosic particles.

Furthermore, the invention provides kits and methods for converting a conventional petroleum petrochemical refinery process for catalytically cracking a triglyceride-containing biomass. In one example, a kit includes instructions for adapting a first conventional petroleum reactor for catalytically cracking triglycerides in a biomass-catalyst mixture, at a temperature below about 300° C., to produce a first oil and a cellulosic portion. The kit also includes instructions for adapting the second conventional petroleum reactor for catalytically cracking cellulose in the cellulosic portion, at a temperature between about 300 and about 600° C., to produce a second oil. In another example, a method includes adapting a first conventional petroleum reactor for catalytically cracking triglycerides in a biomass-catalyst mixture, at a temperature below about 300° C., to produce a first oil and a cellulosic portion. The method also includes adapting the second conventional petroleum reactor for catalytically cracking cellulose in the cellulosic portion, at a temperature between about 300 and about 600° C., to produce a second oil.

Accordingly, the methods, apparatuses, kits, and compositions are useful for facilitating the processing of triglyceride containing biomass. The methods, apparatuses, kits, and compositions can facilitate adapting existing refinery process units for processing biomass (e.g., change operating parameters and feedstock), converting existing refinery process units for processing biomass (e.g., adding an extra riser for biomass), and constructing new, customized biomass reactors (e.g., employ commercially available conventional petroleum reactor components). Thus, the methods, apparatuses, kits, and compositions can reduce the cost and increase the availability of fuel and/or specialty chemicals derived from triglyceride containing biomass.

The methods can have a synergistic effect, reducing the temperature (and, in some cases the pressure) necessary for catalytic conversion of the biomass and/or increasing the conversion efficiency of the biomass and/or facilitating processing of biomass. For example, catalysts can facilitate catalytic conversion under less severe conditions (e.g., lower temperatures and/or shorter time) and with a more efficiency (e.g., higher conversion of the biomass and better quality products from the conversion) than can be achieved in conventional petroleum refinery process units. In some embodiments, the synergistic effect can include increasing the yield of organic compounds usable as a fuel, feedstock, and/or specialty chemical, and/or reducing the yield of undesirable products such as tars and/or unconverted biomass in conventional petroleum refinery process units. In certain embodiments, the synergistic effect can include converting different biomass components (e.g., cellulose, hemicellulose and/or lignin) under milder conditions (e.g., lower temperature than conventional petroleum catalytic cracking) in conventional petroleum refinery process units. The synergistic effect can also include making the products of catalytic conversion more uniform, or increasing the selectivity or proportion of the production of desired products (e.g., increasing the proportion of a fraction usable as a fuel, feedstock, or specialty chemical). These results can be accomplished utilizing equipment already present in conventional petroleum refinery process units.

### Triglyceride Containing Biomass

In various embodiments, biomass (e.g., aquatic biomass, oil seed, or other triglyceride containing biomass) includes materials of photosynthetic (e.g., plant) origin, lipids (e.g., triglycerides, diglycerides, monoglycerides, and aliphatic hydrocarbons), as well as carbohydrates (e.g., lignin, amorphous hemi-cellulose, and crystalline cellulose) and polypeptides (e.g., proteins). The invention can convert the macromolecular components of the biomass, including lipids, carbohydrates, and polypeptides, into fuels and specialty chemicals. Some aquatic plants include little or no lignin. However, the invention is applicable to any biomass including any amount of cellulose, hemicellulose, and/or lignin. Examples of such biomass include, but are not limited to, micro algae (e.g., diatoms and green, blue-green, and golden algae), macro algae (e.g., seaweed), other aquatic plants, oil seed (soy, palm, rape, sunflower seed, peanut, cotton seed, palm kernel, olive, corn, nut, linseed, rice bran, safflower, sesame, and coconut), and the like. Terrestrial plant oil sources also include castor, hemp, mustard, radish, ramtil, tung, copaiba, honge, jatropha, milk bush, and petroleum nut.

Algae are a good source for biomass because they generally have high growth rates, high efficiency (i.e., convert more soar energy into biomass than terrestrial plants), and high hardiness (i.e., can grow under more adverse conditions than terrestrial plants, such as salt water). Algae also do not require the use of arable land, and thus do not necessarily compete with farming foodstuffs. Exemplary macromolecule compositions of various algae are provided in Table 1.

TABLE 1
Chemical Composition of Algae (% Dry Matter)

| | | Carbohy- | | Nucleic |
|---|---|---|---|---|
| Strain | Protein | drates | Lipids | acid |
| Scenedesmus obliquus | 50-56 | 10-17 | 12-14 | 3-6 |
| Scenedesmus quadricauda | 47 | — | 1.9 | — |
| Scenedesmus dimorphus | 8-18 | 21-52 | 16-40 | — |
| Chlamydomonas rheinhardii | 48 | 17 | 21 | — |
| Chlorella vulgaris | 51-58 | 12-17 | 14-22 | 4-5 |
| Chlorella pyrenoidosa | 57 | 26 | 2 | — |
| Spirogyra sp. | 6-20 | 33-64 | 11-21 | — |
| Dunaliella bioculata | 49 | 4 | 8 | — |
| Dunaliella salina | 57 | 32 | 6 | — |
| Euglena gracilis | 39-61 | 14-18 | 14-20 | — |
| Prymnesium parvum | 28-45 | 25-33 | 22-38 | 1-2 |
| Tetraselmis maculata | 52 | 15 | 3 | — |
| Porphyridium cruentum | 28-39 | 40-57 | 9-14 | — |
| Spirulina platensis | 46-63 | 8-14 | 4-9 | 2-5 |
| Spirulina maxima | 60-71 | 13-16 | 6-7 | 3-4.5 |
| Synechoccus sp. | 63 | 15 | 11 | 5 |
| Anabaena cylindrica | 43-56 | 25-30 | 4-7 | — |

The biomass feedstock can include a slurry of aquatic biomass. The slurry can be obtained by collecting algae and subjecting the algae to a dewatering step (e.g., passing the algae over a screen and allowing water to drain by the action of gravity). In one embodiment, the slurry can contain about 10 to about 35 wt % water. In various embodiments, water can be removed by mechanical action (e.g., kneading) or other physical action (e.g., cyclonic or centrifugal force). For example, water can be removed in disintegrating (e.g., mechanical size reduction), agitating, and/or kneading steps. Water can be removed by screening or filtering (e.g., gravity or press filtering. In some embodiments, water can be removed using passive (e.g., drying in the sun) or active (e.g., heating) evaporation, or by other known drying techniques.

The biomass feedstock can be subjected to a particle size reduction step. In various embodiments, the particle size reduction step includes disintegration. In some embodiments, the particle size reduction step includes agitation. In certain embodiments, the particle size reduction step includes kneading. The particle size reduction step can be selected based upon the source of the biomass. For example, more fibrous biomass (e.g., seaweed or oil seed) can require additional or prolonged treatment, to break down the cellulose. The particle size reduction step can also break down cells in the biomass, to make available cellular components (e.g., glycerides).

The biomass feedstock can be combined with a catalyst (described below). For example, the biomass feedstock can be combined with catalysts before, during, and/or after the size reduction step. In various embodiments, the size reduction step can impregnate the catalyst into the biomass and/or facilitate the formation of a mechano-chemical interaction between the catalyst and the biomass.

Biomass sources can be used without requiring refining (e.g., chemically altering the biomass). In various embodiments, biomass includes unrefined or virgin material of photosynthetic origin. Biomass sources can be subjected to a drying and/or a particle size reduction step. Such a drying and/or a particle size reduction step does not significantly change the relative composition of the biomass in terms of cellulose, hemicellulose and/or lignin and therefore such a step is not necessarily considered refining.

In various embodiments, biomass feedstock can include particles that are solid and in a finely divided form (e.g., saw dust and ground straw). Biomass feedstock can include solid materials as well as materials that might be classified as liquids, but that have a very high viscosity (e.g., small or large colony algae). Biomass particles can be prepared from biomass sources and larger particles by techniques such as milling, grinding, pulverization, and the like. Conventional paper processing/pulping methods and equipment can be used to prepare biomass particles. For example, biomass from sources such as straw and wood can be converted to particles in a size range of about 5 mm to about 5 cm using techniques such as milling or grinding.

### Agitation of Biomass Particles

In various embodiments, the method includes agitating solid biomass particles, to reduce a size characterizing at least a portion of the particles. In some embodiments, agitating is facilitated by fluid conveyance, including, without limitation, by gas flow or pneumatic conveyance. Agitating can be conducted in a vertical vessel, such as a riser or downer. An agitator can include a conveyor, a riser, or downer. A riser (up flow) or a downer (down flow) can be, for example, a hollow vertical vessel terminating in a larger diameter vessel, which houses high velocity (e.g., about 60-80 fps) cyclones that may or may not be physically connected to the riser termination point. The height can of a riser or downer can be, for example, between about 15 ft and about 60 ft and the diameter can be, for example, between about 1 ft and about 4 ft. Agitating can be facilitated by a gas (e.g., gas can convey the particles such that they are abraded or ground by other particles, catalyst, and/or inorganic particulate material). The gas can be one or more of air, steam, flue gas, carbon dioxide, carbon monoxide, hydrogen, hydrocarbons, and methane. The gas can be a gas having a reduced level of oxygen (compared to air) or can be substantially oxygen-free. In another embodiment, an agitator can be a kneader or mixer (e.g., for mechanical, as opposed to pneumatic, agitation).

In certain embodiments, agitating includes causing the solid biomass particles to be conveyed at a velocity of greater than about 1 m/s. For example, the velocity can be measured relative to a vessel in which the particles are conveyed. Agitating can include causing the solid biomass particles to move at a velocity of greater than about 10 m/s. Agitating can include causing at least a portion of the solid biomass particles to move at a velocity of greater than about 100 m/s. An agitator can be adapted to cause the solid biomass particles to move at a velocity of greater than about 1 m/s, greater than about 10 m/s, and/or greater than about 100 m/s. Other velocities include velocities of greater than about 5, 25, 50, 75, 125, 150, 175, 200, 225, and 250 m/s.

For example, the velocity is selected from the group consisting of: between about 10 and about 20 m/s; between about 20 and about 30 m/s; between about 30 and about 40 m/s; between about 40 and about 50 m/s; between about 50 and about 60 m/s; between about 60 and about 70 m/s; between about 70 and about 80 m/s; between about 80 and about 90 m/s; and between about 90 and about 100 m/s. The velocity can be about 10 m/s, about 20 m/s, about 30 m/s, about 40 m/s, about 50 m/s, about 60 m/s, about 70 m/s, about 80 m/s, about 90 m/s, or about 100 m/s. The velocity can be greater than about 10 m/s, about 20 m/s, about 30 m/s, about 40 m/s, about 50 m/s, about 60 m/s, about 70 m/s, about 80 m/s, about 90 m/s, or about 100 m/s.

In various embodiments, agitating solid biomass particles, to reduce a size characterizing at least a portion of the particles, is facilitated by agitating solid biomass particles together with a material that is harder than the biomass. For example, the material can be a catalyst or another inorganic particulate material. The amount of size reduction, and thus the size of the resulting solid biomass particles can be modulated by the duration of agitation and the velocity of agitation. Other factors such as the relative hardness of the catalyst of another inorganic particulate material, the dryness (e.g., brittleness), and the method/vessel(s) in which agitation occurs also modulate the amount of size reduction.

In embodiments using an abrading or grinding material that is a catalyst, the catalyst can become embedded in the biomass particles, which can facilitate catalytic conversion of the biomass. In such embodiments, agitating can facilitate formation of a mechano-chemical interaction between at least a portion of the catalyst and at least a portion of the solid biomass particles, which can facilitate catalytic conversion of the biomass.

Agitation can be carried out at an elevated temperature, for drying the biomass. An elevated temperature can be a temperature sufficient to dry the biomass, for example, between about 50 and about 150° C. Higher temperatures can be used, for example, where an agitating gas is oxygen-poor or substantially oxygen-free. Agitation can also be carried out at ambient temperature with dried biomass. Drying increases the hardness of the biomass particles, making the particles more susceptible to size reduction.

Agitation can be carried out by various different methods and in various different vessels. For example, in order of increasing abrasion, the agitation can be carried out in a fluid bed, a bubbling or ebullient bed, a spouting bed, or a conveyor. In one embodiment, agitation is carried out by fluid conveyance, including without limitation by gas flow or pneumatic conveyance. In one embodiment, agitation is carried out in a riser or a downer.

Agitating solid biomass particles, to reduce a size characterizing at least a portion of the particles, can result in a dispersion of particle sizes. For example, proper agitation the solid biomass particles as described above can result in individual particles sizes ranging from microns, to tens of microns, to tenths of centimeters, to centimeters or greater. In various embodiments, at least a fraction of the biomass particles are reduced to a size below about 50, 45, 40, 35, 30, 25, 20, 15, 10, or 5 microns. In one embodiment, at least a fraction of the biomass particles are reduced to a size below about 15 microns. In one embodiment, at least a fraction of the biomass particles are reduced to a size below about 10 microns.

In various embodiments, the plurality of solid biomass particles are substantially characterized by an average size between about 50 and about 70 microns and individual sizes between about 5 and about 250 microns. In other embodiments, the plurality of solid biomass particles are substantially characterized by an average size between about 10 and about 20 microns and individual sizes between about 5 and about 50 microns. In other embodiments, the plurality of solid biomass particles are substantially characterized by an average size between about 100 and about 150 microns and individual sizes between about 5 and about 500 microns.

International Publication No. WO 2007/128798 A1 by O'Connor, the disclosure of which is incorporated herein by reference in its entirety, discloses agitating solid biomass particles and catalysts. In particular, paragraphs [0027] to [0072] of WO 2007/128798 A1 are incorporated herein by reference.

International Publication No. WO 2008/009643 A2 by O'Connor, the disclosure of which is incorporated herein by reference in its entirety, discloses agitating solid biomass particles and catalysts. In particular, paragraphs [0009] to [0051] of WO 2007/128798 A1 are incorporated herein by reference.

### Solid Biomass Particles

Biomass sources can be used without requiring chemical pre-processing (e.g., chemically altering the biomass). In various embodiments, biomass sources include (chemically) unrefined material of photosynthetic origin. Biomass sources can be subjected to a drying and/or a particle size reduction step. Such a drying and/or a particle size reduction step does not significantly change the relative composition of the biomass in terms of cellulose, hemicellulose and/or lignin and therefore such a step is not necessarily considered refining. Solid biomass particles can include biomass solids mixed with oils (e.g., particles produced from oil seed or algae having about the same macromolecular ratio as before particle production). In some embodiments, solid biomass particles are produced from residual solids after an oil extraction step (e.g., from solids remaining after an extraction of oil from oil seed or algae).

In various embodiments, biomass feedstock can include particles that are solid and in a finely divided form (e.g., saw dust and ground straw). Biomass feedstock can include solid materials as well as materials that might be classified as liquids, but that have a very high viscosity (e.g., small or large colony algae). Biomass particles can be prepared from biomass sources and larger particles by techniques such as milling, grinding, pulverization, and the like. For example, biomass from sources such as straw and wood can be converted to particles in a size range of about 5 mm to about 5 cm using techniques such as milling or grinding.

The biomass can be subjected to a particle size reduction step, or can be collected in the form of particles (e.g., algae cells, colonies, flocculated algae, and the like). In various embodiments, the biomass particles are reduced to, or have, an average particle size of less than about 1000 microns. Alternatively, the biomass particles are reduced to, or have, an average particle size of greater than about 1000 microns. The plurality of solid biomass particles can be substantially characterized by individual sizes below about 2000, 1900, 1800, 1700, 1600, 1500, 1400, 1300, 1200, 1100, 1000, 900, 800, 700, 600, 500, 400, 300, 200, or 100 microns. In various embodiments, at least a fraction of the biomass particles have a size of about 1-2000, 1-1500, 1-1000, or 1000-2000 microns. For example, the biomass particles can have an average size of less than about 2000, 1750, 1500, 1250, 1000, 750, 500, or 250 microns. In some embodiments, at least a fraction of the biomass particles are reduced to a size below about 500, 475, 450, 425, 400, 375, 350, 325, 300, 275, 250, 225, 200, 175, 150, 125, 100, 90, 80, 70, 60, 50, 40, 30, 20, 15, 10, or 5 microns. Individual particles sizes can range from microns, to tens of microns, to tenths of centimeters, to centimeters or greater.

Solid biomass particles do not necessarily assume a spherical or spheroid shape. For example, solid biomass particles can be needle shaped and/or assume another cylinder-like or elongated shape. Accordingly, size does not necessarily correspond to a single diameter (although it could correspond to an average diameter or diameter in a singe, for example largest or smallest, dimension). In various embodiments, size can correspond to the mesh size or a screen size used in separation and/or sizing the solid biomass particles.

### Separation of Biomass Particles

In various embodiments, methods include separating a biomass-catalyst mixture into a fine fraction and a coarse fraction. The biomass-catalyst mixture includes the particles and a catalyst. The fine fraction includes particles of about a predetermined size. The coarse fraction includes particles of greater than about the predetermined size. Separating the mixture into a fine fraction and a coarse fraction can have several effects. For example, a fine fraction can be selected to include particles of about a predetermined size, below about a predetermined size, and/or within a predetermined size range. In some embodiments, the fine fraction can be selected to consist essentially of particles of about a predetermined size, below about a predetermined size, and/or within a predetermined size range. Furthermore, a coarse fraction can be recycled for further size reduction and/or to produce more of a fine fraction.

A predetermined size can be selected based upon one or more requirements of a subsequent reaction. For example, a predetermined size can be selected to facilitate substantial catalytic conversion of the fine fraction in a subsequent reaction. A predetermined size can be selected to facilitate contact, impregnation, and/or interaction of the catalyst and the biomass. In some embodiments, a predetermined size can be about 2000, 1900, 1800, 1700, 1600, 1500, 1400, 1300, 1200, 1100, 1000, 900, 800, 700, 600, 500, 450, 400, 350, 300, 250, 200, 150, 100, 50, 45, 40, 35, 30, 25, 20, 15, 10, or 5 microns, or any individual value therebetween. In one embodiment, a predetermined size is about 15 microns. In one embodiment, a predetermined size is about 10 microns. A predetermined size can be between about 1 and 2000 microns or between about 5 and about 1000 microns.

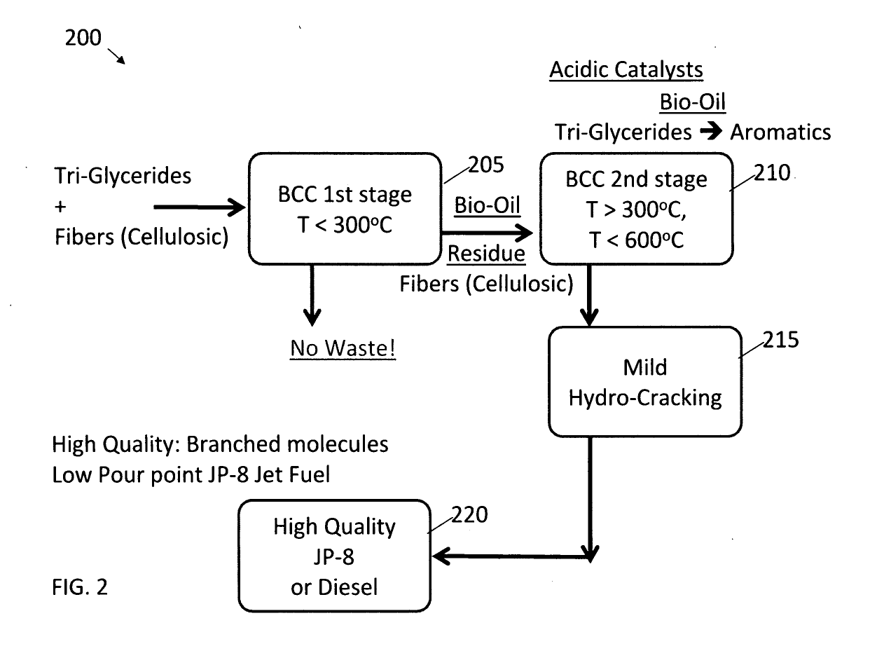

Separating can be facilitated by a cyclonic action. A separator can include a single cyclone. Alternatively, a separator can include a plurality of cyclones arranged, for example, in parallel, series, as a third stage separator, or as a fourth stage separator. U.S. Pat. No. 6,971,594 to Polifka, the disclosure of which is incorporated herein by reference in its entirety, discloses cyclonic action and cyclone separators that can be adapted and employed with the invention. In particular, FIG. 2, the text corresponding to FIG. 2, and the text corresponding to column 4, line 55 to column 11, line 55 of U.S. Pat. No. 6,971,594 is incorporated herein by reference.

Separating can be achieved by other known methods. For example, separating can be achieved by screening, settling, clarification, and the like.

### Catalysts and Inorganic Particulate Materials

A catalyst can be any material that facilitates the conversion of organic components of the biomass into fuels, specialty chemicals, or precursors thereof. In various embodiments, the catalyst includes a solid particulate catalyst and the biomass-catalyst mixture includes at least a portion of the catalyst mechano-chemically interacting with at least a portion of the solid biomass particles. In some embodiments, the catalyst includes a catalyst capable of being at least partly dissolved or suspended in a liquid and the biomass-catalyst mixture includes at least a portion of the catalyst impregnating at least a portion of the solid biomass particles.

In various embodiments, a catalyst is a particulate inorganic oxide. The particulate inorganic oxide can be a refractory oxide, clay, hydrotalcite, crystalline aluminosilicate, layered hydroxyl salt, or a mixture thereof. Suitable refractory inorganic oxides include alumina, silica, silica-alumina, titania, zirconia, and the like. In one embodiment, the refractory inorganic oxides have a high specific surface (e.g., a specific surface area as determined by the Brunauer Emmett Teller (“BET”) method of at least 50 m²/g). Suitable clay materials include cationic and anionic clays, for example, smectite, bentonite, sepiolite, atapulgite, hydrotalcite, and the like. Suitable metal hydroxides and metal oxides include bauxite, gibbsite and their transition forms. Other suitable (and inexpensive) catalysts include lime, brine, and/or bauxite dissolved in a base (e.g., NaOH), or a natural clay dissolved in an acid or a base, or fine powder cement (e.g., from a kiln). Suitable hydrotalcites include hydrotalcite, mixed metal oxides and hydroxides having a hydrotalcite-like structure, and metal hydroxyl salts.

In some embodiments, a catalyst can be a catalytic metal. The catalytic metal can be used alone or together with another catalyst. A catalytic metal can be used in a metallic, oxide, hydroxide, hydroxyl oxide, or salt form, or as a metallo-organic compound, or as a material including a rare earth metal (e.g., bastnesite). In certain embodiments, the catalytic metal is a transition metal. The catalytic metal can be a non-noble transition metal. For example, the catalytic metal can be iron, zinc, copper, nickel, and manganese. In one embodiment, the catalytic metal is iron.

A catalytic metal can be contacted with the biomass by various methods. In one embodiment, the catalyst is added in its metallic form, in the form of small particles. Alternatively, the catalyst can be added in the form of an oxide, hydroxide, or a salt. In another embodiment, a water-soluble salt of the metal is mixed with the biomass and the inert particulate inorganic material in the form of an aqueous slurry. The biomass and the aqueous solution of the metal salt can be mixed before adding the inert particulate inorganic material to facilitate the metal impregnating the biomass. The biomass can also be mixed with the inert particulate inorganic material prior to adding the aqueous solution of the metal salt. In still another embodiment, an aqueous solution of a metal salt is mixed with the inert inorganic material, the material is dried prior to mixing it with the particulate biomass, and the inert inorganic material is thus converted to a heterogeneous catalyst.

The biomass-catalyst mixture can include an inorganic particulate material. An inorganic particulate material can be inert or catalytic. An inorganic material can be present in a crystalline or quasi-crystalline form. Exemplary inert materials include inorganic salts such as the salts of alkali and alkaline earth metals. Although these materials do not necessarily contribute to a subsequent chemical conversion of the polymeric material, it is believed that the formation of discrete particles of these materials within the biomass can work as a wedge to mechanically breaking up or opening the structure of the biomass, which can increase the biomass surface accessible to microorganisms and/or catalysts. In one embodiment, the breaking up or opening is facilitated by crystalline or quasi-crystalline particles.

Inorganic particulate material can have catalytic properties. For example, a catalytic inorganic particulate material can be a metal oxide or hydroxide such as an alumina, silica, silica aluminas clay, zeolite, ionic clay, cationic layered material, layered double hydroxide, smectite, saponite, sepiolite, metal hydroxyl salt, and the like. Carbonates and hydroxides of alkali metals, and the oxides, hydroxides and carbonates of alkali earth metals can also have catalytic properties. Inorganic particulate material can include mixtures of inorganic materials. Inorganic particulate material can include a spent (resid) fluid catalytic cracking catalyst containing (thermally treated) layered material. Employing spent catalyst can involve reusing waste material. The spent catalyst can be ground of pulverized into smaller particles to increasing dispersibility. Inorganic particulate material can also include sandblasting grit. Employing sandblasting grit can involve reusing waste material, which can include particles of iron, and lesser quantities of other suitable metals such as nickel, zinc, chromium, manganese, and the like (e.g., grit from steel sandblasting).

Contacting the catalyst, and optionally the inorganic particulate material, with the biomass, can be achieved by various methods. One method includes heating and fluidizing a mixture of the particulate biomass material and the inert inorganic material, and adding the catalyst to the mixture as fine solid particles. Another method includes dispersing the catalytic material in a solvent (e.g., water), and adding the solvent to the mixture of particulate biomass material and the inert inorganic material.

European Patent Application No. EP 1 852 466 A1 by O'Connor, the disclosure of which is incorporated herein by reference in its entirety, discloses catalysts and contacting catalysts and biomass. In particular, paragraphs [0011] to [0043] of EP 1 852 466 A1 are incorporated herein by reference.

International Publication No. WO 2007/128799 A1 by O'Connor, the disclosure of which is incorporated herein by reference in its entirety, discloses catalysts and contacting catalysts and biomass. In particular, paragraphs [0015] to [0054] of WO 2007/128799 A1 are incorporated herein by reference.

### Pre-Treating Biomass

In various embodiments, biomass feedstock can be chemically and/or physically pre-treated. Examples of pretreatment steps in which recycled aqueous phase can be used include demineralization, heat treatment, and steam explosion.

Demineralization can include removing at least a fraction of a naturally occurring mineral from biomass (e.g., prior to a pyrolysis or catalytic cracking reaction). Demineralization can improve control over the reaction of the biomass. Many of the minerals naturally present in the biomass material can be catalytically active (e.g., potassium, iron). Although these materials can catalyze reactions, they can also increase coke yield, which is generally undesirable. Even when catalytic activity is desired, it can be preferable to first demineralize the biomass material so as to control the composition of their catalyst system.

In various embodiments, a pretreatment can reduce an ash content of biomass, or a hazardous disposal characteristic of an ash that may be subsequently produced. Removal of minerals (e.g., ash precursors) from the biomass can reduce the ash content. Removal of metals (e.g., ash precursors), particularly heavy metals, can also reduce ash content and prevent metal contamination of waste products, thereby facilitating disposal of waste by providing an uncontaminated waste product and reducing the cost of disposing of the waste product.

A pretreatment for reducing ash content can include swelling the biomass with a solvent and then removing solvent from the swollen biomass material by applying mechanical action to the biomass material. Ash precursors, such as dissolved minerals and/or metals, will thus be removed with the solvent. The solvent can be aqueous. The solvent can include an acid or base (e.g., inorganic acid or base). The mechanical action can occur in an agitator and/or a kneader. The mechanical action can be exerted by equipment such as a high shear mixer, kneader, colloid mill, planetary mixer, mix-miller, or ball mill. A pretreatment for reducing ash content can include washing or slurring with an aqueous phase having pH above or below neutral, ion exchange (e.g., with ammonium solutions that would exchange a hydrogen ion with a metal ion), and steam stripping are possible methods. In addition to removing minerals from the biomass, the swelling and dewatering steps can make the biomass material more susceptible to a subsequent reaction.

Although essentially any aqueous solvent can be used for demineralization, the aqueous phase of a liquid pyrolysis product can be particularly effective. The effectiveness is believed to be due to the presence of organic acids (e.g., carboxylic acid, acetic acid) in the aqueous phase. Without wishing to be bound by any theory, the acidity of the aqueous phase can facilitate the mobilization of minerals in the biomass. For example, the chelating effects of carboxylic acids can contribute to the solubilization and removal of mineral cations.

De-mineralizing biomass (e.g., algae) can mitigate at least one of char and ash formation upon conversion (e.g., pyrolysis, catalytic cracking) of the biomass into a fuel or specialty chemical by removing the mineral precursors of the char and/or ash from the biomass. De-mineralizing biomass (e.g., algae) can also produce a fertilizer by separating a fraction of the biomass suitable for use as a fertilizer or specialty chemical. The fraction of the biomass can include a mineral solution as a raw extract (e.g., essentially the solvent removed during de-mineralization) or as a fraction of the raw extract (e.g., water, mineral, or other component at least partially removed).

Pretreatment can reduce ash content to less than about 10 wt %, 9 wt %, 8 wt %, 7 wt %, 6 wt %, 5 wt %, 4 wt %, 3 wt %, 2 wt %, or 1 wt %, based on dry weight of the biomass material. The pretreatment can reduce metal (e.g., Fe) content to less than about 3,000, 2,500, 2,000, 1,500, 1,000, or 500 mg/kg, based on dry weight of the biomass.

Solvent explosion can include contacting the biomass with a pressurized solvent at a temperature above its natural boiling point (e.g., at atmospheric pressure). The pressurized solvent is in a liquid phase and swells the biomass. Then, the solvent is de-pressurized, causing rapid evaporation (e.g., boiling) of the solvent. This rapid evaporation can be referred to as solvent explosion. The solvent explosion can physically rupture the biomass material, thereby making it more accessible in a subsequent reaction.

Examples of solvents that can be used in solvent explosion include ammonia, carbon dioxide, water, and the like. If water is used as the solvent, the process can be referred to as steam explosion. It is understood that the term steam explosion can be considered a misnomer, and that the term water explosion can be more accurate. Nevertheless, the term steam explosion is used herein because it is an accepted term of art. The aqueous phase of the liquid pyrolysis product can be used in a steam explosion.

When steam explosion is combined with demineralization, the steam explosion can be carried out before or after the demineralization. For example, it can be advantageous to conduct the demineralization after the steam explosion because the steam explosion pretreatment can make the minerals more accessible, thereby making the demineralization more effective.

Heat treatment (e.g., torrefaction) can include heating the biomass to a temperature of about 100-300° C. in an oxygen-poor or oxygen-free atmosphere. The term oxygen-poor can refer to an atmosphere containing less oxygen than ambient air. The heat treatment can carried out in the presence of sufficient solvent (e.g., water) to swell the biomass material. The heat treatment can be carried out in a closed vessel to mitigate evaporation of the solvent. In some examples, the vapor (e.g., steam) formed under these conditions can displace oxygen present in the vessel and produce an oxygen-poor atmosphere. In one example, the aqueous phase of a liquid pyrolysis product can be the solvent in such a heat treatment.

Heat treatment can be carried out at a temperature low enough to mitigate carbon loss due to the formation of gaseous conversion products (e.g., CO, CO2). A heat treatment can use, for example, a temperature of about 100-200° C. For example, a temperature can be about 100-140° C. A heat treatment can have a duration, for example, of about 2 min to 2 hours. For example, a duration can be about 20-60 min. In various examples, pressure can be released at the end of a heat treatment by opening the heat treatment vessel, which can allow the heat treatment to be combined with a steam explosion pretreatment step.

Even when the heat treatment essentially does not produce any gaseous conversion products, it can still result in a modification of the biomass. For example, the heat treatment can make the biomass more brittle and more hydrophobic. Both effects can be desirable from the perspective of a subsequent reaction. For example, increased brittleness can facilitate girding the biomass to a small particle size, to increase reactivity in a pyrolysis reaction, and increased hydrophobicity can facilitate drying the biomass.

In one embodiment, a method of producing a biomass feedstock from algae can include torrefying the algae at a temperature below about 300° C., to produce a plurality of solid biomass particles having an increased brittleness and/or susceptibility to catalytic conversion. A heat pretreatment step can be combined with one or more additions pretreatment steps (e.g., demineralization, steam explosion). Because of the increased hydrophobicity of heat treated biomass, it can be preferable to conduct any demineralization and/or steam explosion steps prior to the heat treatment; with the exception that steam explosion can be combined with heat treatment as described above.

### Kneaders

A kneader can be used to knead the solid biomass particles and the catalyst, to make at least a portion of the solid biomass particles accessible to at least a portion of the catalyst. The kneader can be an extruder, miller, mixer, or grinder. The kneader can operate at greater than ambient temperature, for example, to facilitate removal or water and/or other solvent. For example, the kneader can be heated and/or heated gas (e.g., steam) can be provided to heat the biomass and catalyst.

In various embodiments, the kneader employs a solvent. The solvent can be water, an alcohol (e.g., ethanol or glycerol), a bio-oil or another product from the conversion of the biomass, a liquid acid, an aqueous acid or base, liquid CO₂, and the like. In one embodiment, the solvent is water (e.g., added water and/or water inherent in the biomass), which can be selected for its availability, low cost, and/or ease of handling. In another embodiment, the solvent is a liquid produced during the subsequent conversion of the biomass, which can be selected for its availability. A solvent can be selected to improve penetration of a catalyst into biomass. A solvent can also improve penetration of a catalyst into biomass because a dry biomass can be more difficult to penetrate. A solvent can also be selected to remove ash precursors. Solvents can be removed (e.g., by drying) prior to subsequent processing and/or conversion. A kneader can remove at least a portion of a solvent absorbed in a biomass (e.g., by mechanical action and draining). Embodiments employing a kneader and a solvent can reduce the ash and/or mineral and/or metal content of the biomass.

In various embodiments, the biomass can be kneaded with one or more solid catalyst and/or inorganic particulate material. In some embodiments, the biomass can be kneaded with a dissolved and/or suspended catalyst. The dissolved and/or suspended catalyst can be used together with one or more solid catalyst and/or inorganic particulate material. Kneading can be continued and/or repeated to produce a biomass-catalyst mixture having the desired properties (e.g., particle size and/or degree of sensitization).

International Publication No. WO 2007/128800 A1 by O'Connor, the disclosure of which is incorporated herein by reference in its entirety, discloses catalysts and sensitizing biomass, as well as sensitizing by kneading. In particular, paragraphs [0025] to [0074] with respect to catalysts and sensitizing biomass, as well paragraphs [0076] to [0086] with respect to sensitizing by kneading, of WO 2007/128800 A1 are incorporated herein by reference.

### Disintegrators

The disintegrator processes plant matter at a location in close proximity to an agricultural site used to produce such plant matter, to produce the solid biomass particles. In operation, a disintegrator can be used to modify the consistency of, e.g., biomass feedstock, and/or to reduce its average particle size. The disintegrator can include at least one of a mill, fragmenter, fractionator, granulator, pulverizer, chipper, chopper, grinder, shredder, mincer, and a crusher. Apparatuses including a disintegrator can process plant matter at a location in close proximity to an agricultural site used to produce such plant matter, to produce the solid biomass particles. U.S. Pat. No. 6,485,774 to Bransby, the disclosure of which is incorporated herein by reference in its entirety, discloses a method of preparing and handling chopped plant materials. In particular, the text corresponding to column 1, line 45 to column 4, line 65 of U.S. Pat. No. 6,485,774 is incorporated herein by reference.

### EXAMPLES

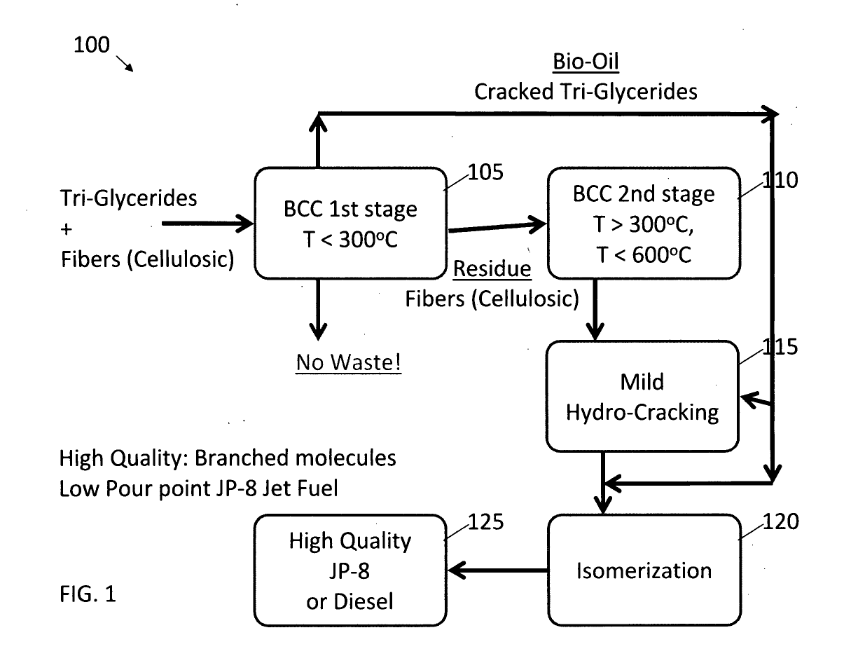

FIGS. 1-6 show exemplary methods for preparing and processing biomass with catalyst. The invention also includes apparatuses for carrying out the methods shown in FIGS. 1-6. The invention also includes kits for setting up the apparatuses and instructions for carrying out the methods corresponding to FIGS. 1-6. The invention also includes products and intermediates, and fractions thereof, corresponding to the methods shown in FIGS. 1-6. It should be understood that one skilled in the art could modify or adapt the exemplary systems, or any other system described herein, to convert biomass into fuels or specialty chemicals. For example, catalyst, reaction vessel(s), pretreatment, and reaction conditions can be selected based upon the type of biomass and the desired product. In some embodiments, the processing can occur in a single vessel. In other embodiments, two or more vessels can be used.

In various embodiments, the intermediates include hydrocarbons from which oxygen is stripped (e.g., as CO, CO₂, H₂O) to produce traditional hydrocarbon products such as light gases, naphtha, heating oils, and the like. In general, processing proceeds by cracking and deoxygenating (as necessary) polymeric compounds in the biomass into hydrocarbon products. In various embodiments, intermediates can be stripped quickly from the catalysts and unconverted biomass to limit secondary (e.g., undesired) reactions.

In various embodiments, the methods do not require steam reforming and/or oligomerization steps to produce desired fuel or specialty chemical products because the methods do not require extensive breakdown and buildup to produce the desired hydrocarbon molecules. Furthermore, methods that produce aromatic compounds, which are subsequently hydro-cracked, do not require a separate isomerization step because they can directly produce branched aliphatic hydrocarbon molecules.

In some embodiments, the methods achieve higher conversion efficiencies than conventional petroleum methods (e.g., known petrochemical refining/production methods). For example, the methods can achieve conversion efficiencies of up to about 55, 60, 65, 70, 75, 80, 85, 90, 95, or 100% with respect to triglycerides, cellulose, and/or overall biomass. In one embodiment, the conversion efficiency for triglycerides is about 85%, the conversion efficiency for cellulose is about 85%, and the overall efficiency is about 72% (i.e., 85%×85%).

In certain embodiments, the methods can be carried out using conventional petroleum equipment. For example, the methods can be carried out in a preexisting petrochemical refinery. The methods can be carried our along with conventional petroleum petrochemical refining. In some embodiments, intermediated and/or products can be co-processed (e.g., with conventional petroleum feedstock, intermediates, products) and/or fed into conventional petroleum refinery process units.

Where an apparatus can co-process a biomass feedstock and a conventional petroleum feedstock, the catalytic cracking system can include a first feed system and a second feed system (not shown). The first feed system can provide the biomass feedstock (e.g., a biomass-catalyst mixture, a plurality of solid biomass particles, at least a portion of a liquefied biomass feedstock) to a refinery unit. The second feed system can provide the conventional petroleum feedstock to a refinery unit. The first feed system can be adapted to provide a suspension of a solid biomass feedstock in a liquefied biomass feedstock or a petroleum feedstock (e.g., torrefied biomass particles suspended in a biocrude or crude oil).

A reactor can operate in either a continuous or switching (e.g., swing reactor) fashion. For example, each train of the refinery unit (e.g., hydroprocessing, hydrocracking unit) can be proceeded by a pair of switchable guard reactors, so that catalyst in the reactor not in operation can be replaced to remove contaminants without allowing a disruptive pressure drop to occur. A guard reactor can include a system for removing and replacing spent catalyst with fresh catalyst (e.g., an ebulating bed reactor with a system to remove spent catalyst and a system to add fresh catalyst). Where the reactor is operated in a continuous fashion, the catalyst can be continuously replaced or regenerated. A guard reactor can help extend catalyst life in the main reactor, by limiting catalyst deactivation due to contaminants substantially to the guard reactor.

In some cases, selecting a biomass feedstock having a relatively low mineral content (e.g., essentially cellulose) or de-mineralizing the biomass feedstock (e.g., by pre-processing) can mitigate the need to replace or regenerate the catalyst. Where the reactor is operated in a switching fashion, it can be important to limit the mineral content of the biomass feedstock to ensure sufficient catalytic activity throughout a reaction cycle. A guard reaction can also be employed to mitigate inactivation of the hydro treating catalyst by minerals in the biomass feedstock. Catalyst (e.g., in a guard reactor) can be selected to have a greater than average macroporous region pore volume, so that it can tolerate a greater quantity of contaminants before becoming inactivated. To some degree, sufficient catalytic activity can be ensured by selecting more active catalyst or providing more catalyst.

The methods can be carried out at lower temperatures than some conventional petroleum methods (e.g., steam reforming, which can require temperatures greater than about 800° C.). In various embodiments, lower temperature can be less than about 300° C. (e.g., for catalytically cracking triglycerides). For example, the lower temperature can be about 300, 275, 250, 225, 200, 175, or 150° C., or between about 300 and 275, 275 and 250, 250 and 225, 225 and 200, 200 and 175, or 175 and 150° C. In various embodiments, lower temperature can be between about 300 and 600° C. (e.g., for catalytically cracking cellulose, or cellulose and triglycerides). For example, the lower temperature can be about 600, 575, 550, 525, 500, 475, 450, 425, 400, 375, 350, 325, or 300° C., or between about 600 and 575, 575 and 550, 550 and 525, 525 and 500, 500 and 475, 475 and 450, 450 and 425, 425 and 400, 400 and 375, 375 and 350, 350 and 325, or 325 and 300° C.

The methods can be carried out at atmospheric pressure. However, in some embodiments, the methods can be carried out a sub-atmospheric pressure (e.g., vacuum pyrolysis). In certain embodiments, the methods can also be carried out a higher than atmospheric pressures (e.g., less than about 4 bar).

FIG. 1 shows a method 100 for catalytically cracking biomass including triglycerides and cellulose. The method includes step 105 of cracking triglycerides, step 110 of cracking cellulose, step 115 of hydro-cracking, step 120 of isomerizing or alkylating, and step 125 of obtaining a fuel or specialty chemical.

Step 105 includes catalytically cracking triglycerides in a triglyceride-rich biomass-catalyst mixture, to produce a first bio-oil. In various embodiments, catalytic cracking occurs at a temperature below about 300° C. Step 105 also produces a cellulosic residue.

In various embodiments, step 105 produces no (or minimal) waste because all of the cellulose in the biomass can also be used as feedstock, rather than being disposed of as waste.

Step 105 also produces no (or minimal) waste because all of the first bio-oil can be used/recycled for further processing/refining and the cellulosic residue, which includes catalyst and can include other components, can be used/recycled for further catalytic cracking. In some embodiments, a vapor can be condensed to form the first bio-oil. At least a portion of the first bio-oil can be separated and send for further processing/refining. At least a portion of the first bio-oil can be recycled for further catalytic cracking.

Step 110 includes catalytically cracking cellulose in the cellulosic residue, to produce a second bio-oil. In various embodiments, catalytic cracking occurs at a temperature between about 300 and about 600° C.

In various embodiments, step 110 has a higher conversion efficiency than known methods for converting triglyceride containing biomass and can use/recycle the first bio-oil for further processing/refining, to increase yield. In some embodiments, waste from the methods is substantially limited to ash. In various embodiments, at least a portion of the catalyst can be recycled. For example, coke deposited on the catalysts can be burnt off, and the heat from combustion can be used for the catalytic cracking steps. In some embodiments, a vapor can be condensed to form the second bio-oil. At least a portion of the second bio-oil can be separated and send for further processing/refining. At least a portion of the second bio-oil, or other solids or liquids, can be recycled for further catalytic cracking.

Step 115 includes hydro-cracking (e.g., mild hydro-cracking) the second bio-oil, to produce a hydro-cracked portion. In various embodiments, step 115 also includes mildly hydro-cracking at least a portion of the first bio-oil, to produce a hydro-cracked portion. In some embodiments, at least a fraction of the hydro-cracked portion can be separated and used as a fuel or specialty chemical.

Step 120 includes isomerizing or alkylating the hydro-cracked portion, to produce at least one fuel (e.g., high-quality JP-8 or diesel) and/or specialty chemical (e.g., branched hydrocarbon molecules). In various embodiments, step 120 can include isomerizing or alkylating at least a portion of the first bio-oil. Isomerizing or alkylating can produce branched molecules. Alternatively, step 120 can include both isomerizing and alkylating at least a portion of the first bio-oil.

In this or any other embodiment including alkylation, alkylating at least a portion of a bio-oil can include adding a C4 or C3 moiety (or, a C1, C2, C5, or greater moiety) to the bio-oil. Alkylation can include alkylating an olefin.

Step 125 includes obtaining a fuel or specialty chemical. For example, step 125 can include separating a fraction from the product stream after isomerization. Step 125 can include purifying a fraction from the product stream after isomerization. In one embodiment, step 125 can be as simple as collecting a fraction of the hydro-cracked portion. Step 125 can also include blending, for example, blending the fraction with a conventionally obtained fuel or specialty chemical.

FIG. 2 shows a method 200 for catalytically cracking biomass including triglycerides and cellulose. The method includes step 205 of cracking triglycerides, step 210 of cracking cellulose, step 215 of hydro-cracking, step 220 of obtaining a fuel or specialty chemical.

Step 205 includes catalytically cracking triglycerides in a triglyceride-rich biomass-catalyst mixture, to produce a first bio-oil. In various embodiments, catalytic cracking occurs at a temperature below about 300° C. Step 205 also produces a cellulosic residue.

In various embodiments, step 205 produces no (or minimal) waste because all of the cellulose in the biomass can also be used as feedstock, rather than being disposed of as waste. Step 205 also produces no (or minimal) waste because all of the first bio-oil can be used/recycled for further processing/refining and the cellulosic residue, which includes catalyst and can include other components, can be used/recycled for further catalytic cracking. In some embodiments, a vapor can be condensed to form the first bio-oil. At least a portion of the first bio-oil can be separated and send for further processing/refining.

Step 210 includes catalytically cracking cellulose in the cellulosic residue, together with the first bio-oil, at a temperature between about 300 and about 600° C. Step 210 can employ an acidic catalyst. Alternatively, step 210 can employ a basic catalyst. Step 210 produces a second bio-oil, which includes aromatic compounds. The aromatic compounds can result from the effect of the higher temperature (compared to step 205) catalytic cracking of the first bio-oil.

In various embodiments, step 210 has a higher conversion efficiency than known methods for converting triglyceride containing biomass and can use/recycle the first bio-oil for further processing/refining, to increase yield. In various embodiments, at least a portion of the catalyst can be recycled. For example, coke deposited on the catalysts can be burnt off, and the heat can be used for the catalytic cracking steps. In some embodiments, a vapor can be condensed to form the second bio-oil. At least a portion of the second bio-oil can be separated and send for further processing/refining. At least a portion of the second bio-oil, or other solids or liquids, can be recycled for further catalytic cracking.

Step 215 includes hydro-cracking (e.g., mild hydro-cracking, replacing O with H) the second bio-oil, to produce a hydro-cracked portion. Hydro-cracking aromatic compounds can produce branched molecules directly from the second bio-oil (e.g., at least a portion of the second bio-oil can be directly converted into branched molecules without requiring a separate isomerization step). In some embodiments, at least a fraction of the hydro-cracked portion can be separated and used as a fuel or specialty chemical.

Step 220 includes obtaining a fuel or specialty chemical. For example, step 220 can include separating a fraction from the product stream after isomerization. Step 220 can include purifying a fraction from the product stream after isomerization. In one embodiment, step 220 can be as simple as collecting a fraction of the hydro-cracked portion. Step 220 can also include blending, for example, blending the fraction with a conventionally obtained fuel or specialty chemical.

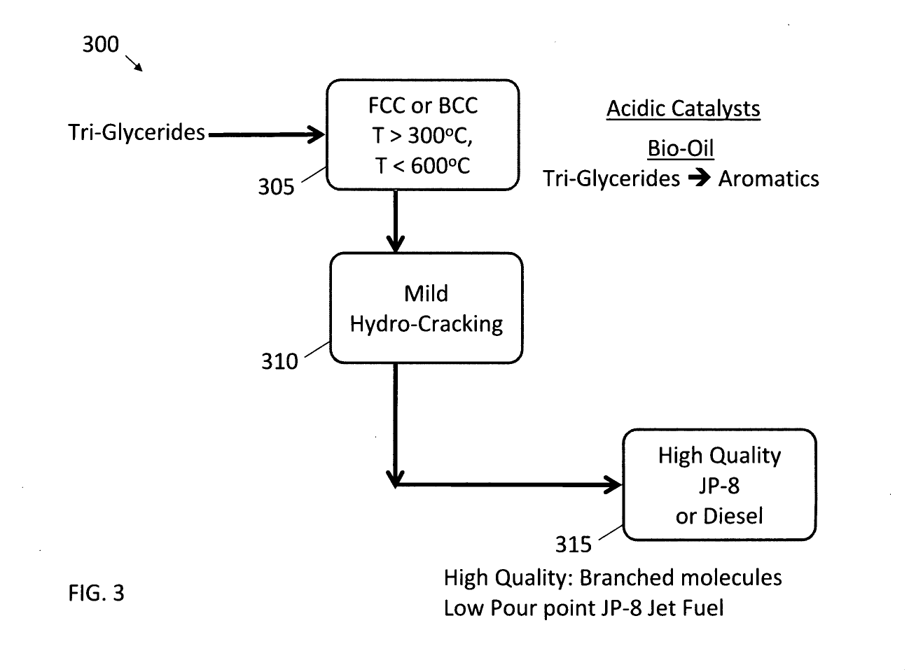

FIG. 3 shows a method 300 for catalytically cracking biomass including triglycerides. The method includes step 305 of catalytic cracking, step 310 of hydro-cracking, and step 315 of obtaining a fuel or specialty chemical.

Step 305 includes catalytically cracking triglycerides in a triglyceride-rich biomass-catalyst mixture, at a temperature between about 300 and about 600° C. Step 305 can employ an acidic catalyst. Step 305 produces a bio-oil, which includes aromatic compounds. The aromatic compounds can result from the effect of the higher temperature catalytic cracking.

In various embodiments, step 305 produces no (or minimal) waste because all of the cellulose in the biomass can also be used as feedstock, rather than being disposed of as waste. Step 305 also produces no (or minimal) waste because all of the bio-oil can be used/recycled for further processing/refining and the cellulosic residue, which includes catalyst and can include other components, can be used/recycled for further catalytic cracking. In various embodiments, at least a portion of the catalyst can be recycled. For example, coke deposited on the catalysts can be burnt off, and the heat can be used for the catalytic cracking steps. In some embodiments, a vapor can be condensed to form the bio-oil. At least a portion of the bio-oil can be separated and send for further processing/refining. At least a portion of the bio-oil, or other solids or liquids, can be recycled for further catalytic cracking.

Step 310 includes hydro-cracking (e.g., mild hydro-cracking, replacing O with H) the bio-oil, to produce a hydro-cracked portion. Hydro-cracking aromatic compounds can produce branched molecules directly from the bio-oil (e.g., at least a portion of the second bio-oil can be directly converted into branched molecules without requiring a separate isomerization step). In some embodiments, at least a fraction of the hydro-cracked portion can be separated and used as a fuel or specialty chemical.

Step 315 includes obtaining a fuel or specialty chemical. For example, step 315 can include separating a fraction from the product stream after isomerization. Step 315 can include purifying a fraction from the product stream after isomerization. In one embodiment, step 315 can be as simple as collecting a fraction of the hydro-cracked portion. Step 315 can also include blending, for example, blending the fraction with a conventionally obtained fuel or specialty chemical.

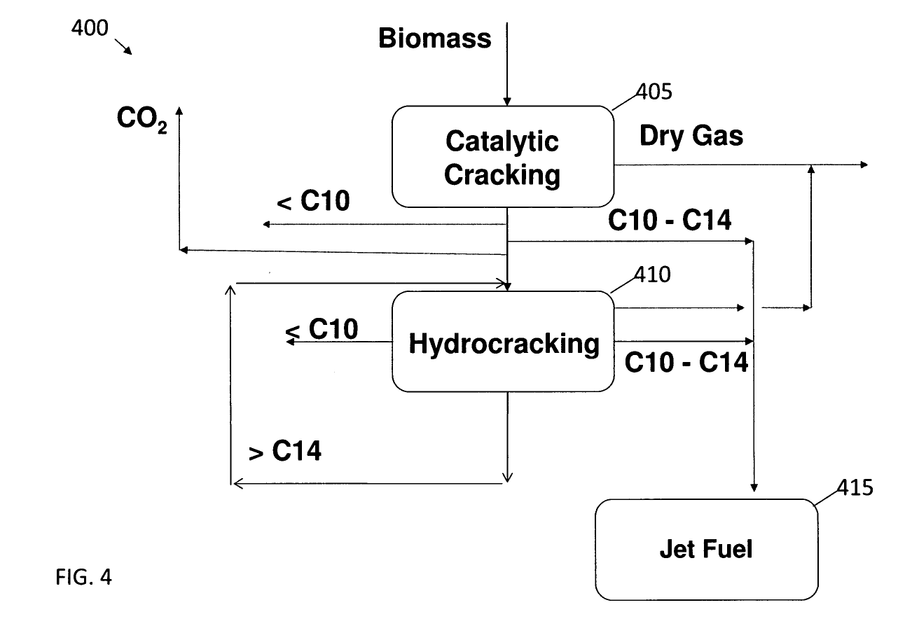

FIG. 4 shows a method 400 for processing a triglyceride-containing biomass without an oil extraction step. The method 400 includes step 405 of catalytic cracking, step 410 of hydrocracking, and step 415 of collecting a product. The triglyceride-containing biomass can be whole algae (or oil seed and the like) that has not been subject to an oil extraction or separation.

Step 405 includes catalytically cracking the biomass (e.g., biomass catalytic cracking, or BCC), to convert the biomass into a liquid product. Catalytic cracking can include any one or more catalytic cracking steps, as described above. For example, catalytic cracking can include any one or more of catalytic cracking steps 105, 110, 205, 210, and 305 described in connection with FIGS. 1-3.

The liquid product includes hydrocarbons that can be separated into one or more fractions for further use, blending, or processing. For example, a fraction can include <C10 hydrocarbons (e.g., C5-C10), for use as gasoline. Lower C hydrocarbons (e.g., <C5) can be oligomerized. A fraction can include C10-C14 hydrocarbons, for use as jet fuel (e.g., JP-8). Higher C hydrocarbons can be hydrocracked (e.g., provided to step 410).

Step 405 can produce a gas such as dry gas (e.g., alcohols, natural gas) and greenhouse gas (e.g., CO₂). CO₂ gas can be provided to a plant (e.g., algae) growth source, to facilitate plant growth.

Step 410 includes hydrocracking a fraction of the liquid product (e.g., mild hydrocracking), to refine the fraction of the liquid product. Hydrocracking can include producing a refined product having a desired number of carbon atoms. For example, hydrocracking can include hydrocracking >C14 hydrocarbons, to produce <C10 hydrocarbons (e.g., C5-C10, for use as gasoline) and C10-C14 hydrocarbons (e.g., for use as jet fuel, JP-8). >C14 hydrocarbons can be recycled for further hydrocracking.

Step 410 can produce a gas such as dry gas (e.g., alcohols, natural gas) and greenhouse gas (e.g., CO₂). CO₂ gas can be provided to a plant (e.g., algae) growth source, to facilitate plant growth.

Step 415 includes collecting jet fuel (e.g., C10-C14 hydrocarbons) from one or more of steps 405 and 410. The collected jet fuel (from biomass) can be used independently or blended with a refinery jet fuel stream (from petroleum).

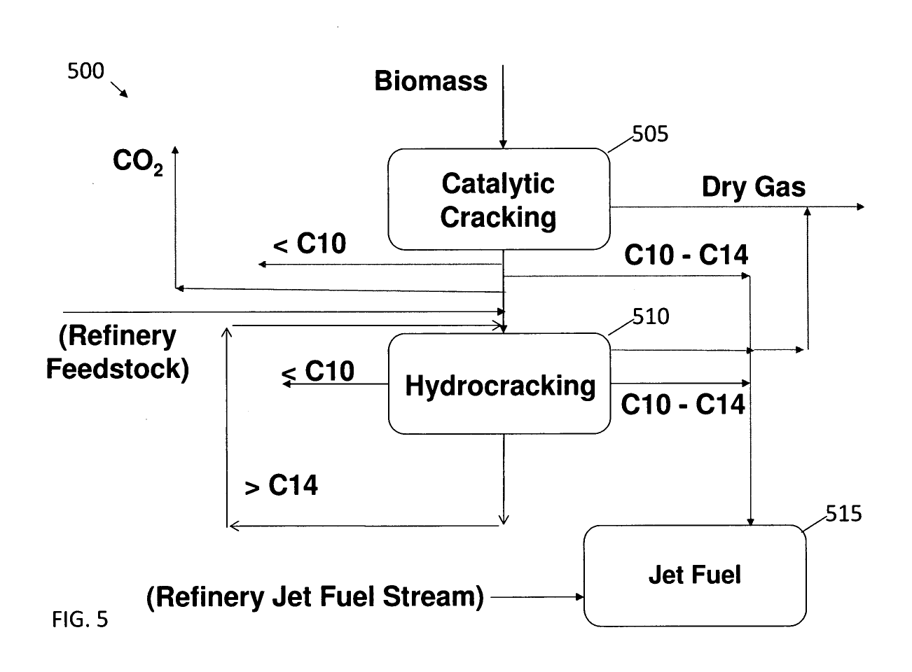

FIG. 5 shows a method 500 for co-processing a refinery feedstock and a triglyceride-containing biomass without an oil extraction step. The method 500 includes step 505 of catalytic cracking, step 510 of hydrocracking, and step 515 of collecting a product. The triglyceride-containing biomass can be whole algae (or oil seed and the like) that has not been subject to an oil extraction or separation. The refinery feedstock can include any one or more of a refinery distillate, an atmospheric gas oil or a vacuum gas oil from a paraffinic or naphthenic crude source, a resid from a paraffinic or naphthenic crude source, a hydrotreated vacuum gas oil, a hydrotreated resid, and a hydrotreated light cycle oil, gasoline, light cycle gas oil, light gas, light crude oil, natural gas, diesel, petroleum coke, liquefied petroleum gas, jet fuel, and the like.

Step 505 of catalytic cracking can include any one or more of the features of step 405. A fraction of the liquid product produced in step 505 can be selected to be co-processed with a refinery feedstock.

Step 510 of hydrocracking can include any one or more of the features of step 410. A refinery feed stock can be provided with, or blended with, a fraction of the liquid product and co-processed with the fraction. In some embodiments, the refinery feedstock is selected to chemically complement the fraction (e.g., the refinery feed stock can act as a hydrogen donor and the fraction as a hydrogen acceptor during hydrocracking) In some embodiments, the fraction can be blended with the refinery feedstock to attain a predetermined fraction of renewable carbon in the product (e.g., jet fuel having greater than 10, 20, 30, 40, or 50% carbon from a renewable source).

Step 515 of collecting a product can include any one or more of the features of step 415. In some embodiments, step 415 includes the optional step of receiving a refinery jet fuel stream, and blending the collected jet fuel (from biomass) with the refinery jet fuel stream (from petroleum). Such blending can produce a product having a predetermined fraction of renewable carbon (e.g., jet fuel having greater than 10, 20, 30, 40, or 50% carbon from a renewable source).

While the invention has been particularly shown and described with reference to specific embodiments, it should be understood by those skilled in the art that various changes in form and detail may be made without departing from the spirit and scope of the invention as defined by the appended claims.

What is claimed is:

1. A method for producing high-quality JP-8 or diesel fuel from a triglyceride-rich biomass such as algae or oil seed, the method comprising:
catalytically cracking triglycerides in a triglyceride-rich biomass-catalyst mixture, at a temperature below about 300° C., to produce a first bio-oil and a cellulosic residue;
catalytically cracking cellulose in the cellulosic residue, at a temperature between about 300 and about 600° C., to produce a second bio-oil;
mildly hydro-cracking the second bio-oil and, optionally, mildly hydro-cracking at least a portion of the first bio-oil, to produce a hydro-cracked portion; and
isomerizing or alkylating the hydro-cracked portion and, optionally isomerizing or alkylating at least a portion of the first bio-oil, to produce high-quality JP-8 or diesel fuel.

2. A method for producing high-quality JP-8 or diesel fuel from a triglyceride-rich biomass such as algae or oil seed, the method comprising:
catalytically cracking triglycerides in a triglyceride-rich biomass-catalyst mixture, at a temperature below about 300° C., to produce a first bio-oil and a cellulosic residue;
catalytically cracking cellulose in the cellulosic residue, at a temperature between about 300 and about 600° C., together with the first bio-oil and an acidic or basic catalyst, to produce a second bio-oil comprising aromatics; and
mildly hydro-cracking the second bio-oil, to produce high-quality JP-8 or diesel fuel.

3. A method for producing high-quality JP-8 or diesel fuel from a triglyceride-rich biomass such as algae or oil seed, the method comprising:
catalytically cracking triglycerides in a triglyceride-rich biomass-catalyst mixture, at a temperature between about 300 and about 600° C., wherein the catalyst comprises an acidic catalyst, to produce a bio-oil comprising aromatics; and
mildly hydro-cracking the bio-oil, to produce high-quality JP-8 or diesel fuel.

4. A method for catalytically cracking a triglyceride-containing biomass, the method comprising:
catalytically cracking triglycerides in a biomass-catalyst mixture, at a temperature below about 300° C., to produce a first oil and a cellulosic portion; and
catalytically cracking cellulose in the cellulosic portion, at a temperature between about 300 and about 600° C., to produce a second oil.

5. The method of claim 4, further comprising:
separating the first oil from the cellulosic portion before catalytically cracking cellulose in the cellulosic portion; and
hydro-cracking the first oil and the second oil, to produce a hydro-cracked product.

6. The method of claim 5, further comprising isomerizing or alkylating the hydro-cracked product, to produce a JP-8 or diesel fuel.

7. The method of claim 4, further comprising:
separating the first oil from the cellulosic portion before catalytically cracking cellulose in the cellulosic portion; and
hydro-cracking the second oil, to produce a hydro-cracked product.

8. The method of claim 7, further comprising isomerzing the first oil and the hydro-cracked product, to produce a JP-8 or diesel fuel.

9. The method of claim 4, further comprising catalytically cracking cellulose in the cellulosic residue, together with the first bio-oil and an acidic or basic catalyst, to produce a second bio-oil comprising aromatics.

10. The method of claim 9, further comprising hydro-cracking the second oil, to produce a JP-8 or diesel fuel.

11. A method for catalytically cracking a triglyceride-containing biomass, the method comprising catalytically cracking triglycerides in a biomass-catalyst mixture, at a temperature between about 300 and about 600° C., wherein the catalyst comprises an acidic or basic catalyst, to produce an oil comprising aromatics.

12. The method of claim 11, further comprising hydro-cracking the oil, to produce JP-8 or diesel fuel.

13. A method for converting a conventional petrochemical refinery for catalytically cracking a triglyceride-containing biomass, the method comprising:
providing or adapting a first conventional petroleum reactor for catalytically cracking triglycerides in a biomass-catalyst mixture, at a temperature below about 300° C., to produce a first oil and a cellulosic portion; and
providing or adapting the second conventional petroleum reactor for catalytically cracking cellulose in the cellulosic portion, at a temperature between about 300 and about 600° C., to produce a second oil.

14. The method of claim 13, further comprising providing a system for separating the first oil from the cellulosic portion and for providing the first oil for mild hydro-cracking or isomerization.

15. The method of claim 4, further comprising catalytically cracking any one or more of a protein, nucleic acid, lignin or other non-cellulosic carbohydrate, and non-triglyceride lipid.
